(19) United States
(12) Patent Application Publication
(10) Pub. No.: US 2023/0326553 A1
(43) Pub. Date: Oct. 12, 2023

(54) IDENTIFYING A TARGET NUCLEIC ACID

(71) Applicant: IMPERIAL COLLEGE INNOVATIONS LIMITED, London (GB)

(72) Inventors: Jesus RODRIGUEZ MANZANO, London (GB); Ahmad MONIRI, London (GB); Luca MIGLIETTA, London (GB); Pantelis GEORGIOU, London (GB)

(21) Appl. No.: 18/042,285

(22) PCT Filed: Aug. 20, 2021
(86) PCT No.: PCT/EP2021/073184

§ 371 Date: Feb. 20, 2023

(30) Foreign Application Priority Data
Aug. 20, 2020 (GB) 2013035.7

(51) Int. Cl. G16B 25/00; G16B 40/20; G06N 3/045; G06N 3/09

(52) U.S. Cl. CPC G16B 25/00; G06N 3/09; G06N 3/045; G16B 40/20

(57) ABSTRACT

Disclosed herein is a computer-implemented method of identifying the presence of any of a plurality of prospective target nucleic acids in a solution containing a biological sample. The method comprises receiving amplification curve data indicative of an amplification reaction associated with at least one unknown nucleic acid present in the solution; processing the received data, wherein the processing comprises inputting input data into a machine learning model trained to identify any of the plurality of prospective target nucleic acids, wherein the input data is based on the amplification curve data and is indicative of the degree of amplification of the at least one unknown nucleic acid over time during the amplification reaction; and based on the processing, determining that the at least one unknown nucleic acid is one of the plurality of prospective nucleic acids, and thereby identifying the presence of at least one of the plurality of target nucleic acids in the solution.

This disclosure relates to identifying the presence of at least one target nucleic acid, and in particular to identifying the presence of any of a plurality of prospective target nucleic acids in a solution containing a biological sample.

### BACKGROUND

There is a need to identify target nucleic acids (such as bacteria, viruses, fungi, genetic variants related to cancer etc.) present in a biological sample, in particular for diagnostic purposes. There is a need to increase the diagnostic throughput associated with this identification, e.g. to enable the identification of more target nucleic acids more quickly. It would be further advantageous to enable this type of identification with less cost and without the need for large laboratory equipment. These factors are important across many applications, such as detecting infectious diseases or preventing the misuse of antibiotics.

Multiplex reactions enable the amplification of several different nucleic acids simultaneously, with the aim of identifying one or more different target nucleic acids. This approach increases diagnostic throughput, and as the need for high throughput analysis of multiple targets continues to escalate, several approaches have been proposed to simultaneously detect and quantify multiple nucleic acids. However, prior approaches have several disadvantages. To date, multiplexing assays have relied on: fluorescent probes (e.g. TaqMan), post-amplification processing (e.g. melting curve analysis, gel electrophoresis or sequencing) or extracting features of the real-time amplification data (e.g. final fluorescent intensity).

Recently, in qPCR, it was shown that sufficient information exists within the amplification curve so as to distinguish several targets using multidimensional standard curves. However, since the volume of data from qPCR is limited (<10² reactions per experiment), explicit features of the amplification curve were extracted to perform reliable multiplexing in a single-channel. While this approach is successful in multiplexing to a degree, it would be desirable to improve upon this prior method's ability to reliably distinguish between different target nucleic acids.

It is desirable to provide a method which offers an affordable solution for detecting multiple nucleic acids, preferably in a single chemical reaction, with increased accuracy, reliability and scalability.

The present invention seeks to address these and other disadvantages encountered in the prior art by providing an improved method of identifying the presence of any of a plurality of prospective target nucleic acids in a solution containing a biological sample.

### SUMMARY

According to an aspect, there is provided a computer-implemented method of identifying the presence of any of a plurality of prospective target nucleic acids in a solution containing a biological sample. The method comprises receiving amplification curve data indicative of an amplification reaction associated with at least one unknown nucleic acid present in the solution. The received data is then processed, the processing comprising inputting data into a machine learning model trained to identify any of the plurality of prospective target nucleic acids. The input data is based on the amplification curve data and is indicative of the degree of amplification of the unknown nucleic acid over time during the amplification reaction. Based on the processing, it is determined that the unknown nucleic acid is one of the plurality of prospective nucleic acids, and the presence of at least one of the plurality of target nuclei is identified in the solution.

The amplification curve data may be received from a thermocycler or a device configured to perform an amplification reaction. The receiving of data and processing of said data may occur in real-time as the amplification reaction is ongoing.

The amplification curve data and/or the input data may comprise a time series depicting the degree of amplification over time throughout a majority of the duration of the amplification reaction. The time series may depict the degree of amplification throughout the entirety of the duration of the amplification reaction. The amplification curve data and/or the input data may comprise a time series depicting the degree of amplification over time from an initial phase in which no amplification is occurring until at least a saturation phase. The amplification curve data and/or the input data may be representative of an entire amplification curve.

The amplification curve data may be real-time PCR data. The amplification curve data may further be real-time digital PCR data.

The method may further comprise pre-processing the amplification curve data to generate the input data, wherein pre-processing may comprise any of background subtraction and normalization.

The machine learning model may have been trained using labelled amplification curve data comprising respective data subsets, each associated with a different one of the plurality of prospective target nucleic acids.

The method may further comprise determining, based on the processing, which of the plurality of prospective target nucleic acids the unknown nucleic acid is most likely to be.

The method may further comprise receiving melting curve data associated with the at least one unknown nucleic acid, the melting curve data being indicative of a degree of dissociation of the at least one unknown nucleic acid with increasing temperature. The input data may further be based on the melting curve data. The machine learning model may have been trained using labelled melting curve data comprising respective data subsets, each associated with a different one of the plurality of prospective target nucleic acids. The degree of dissociation of the at least one unknown nucleic acid may be determined via monitoring the fluorescence of the solution. The solution may contain an intercalating dye.

The input data may be combined input data, and the machine learning model may be a concluding machine learning model in a system of machine learning models comprising a first, a second, and the concluding machine learning model. Processing the received data may further comprise inputting first input data into the first machine learning model. The first input data may be based on the received amplification curve data and the first machine learning model may be trained to identify any of the plurality of prospective target nucleic acids based on the first input data. The second input data may be input into the second machine learning model. The second input data may be based on the received melting curve data and the second machine learning model may be trained to identify any of the plurality of prospective target nucleic acids based on the second input data. The combined input data may be generated based on outputs from the first and second machine learning models. The combined input data may be input into the concluding machine learning model, the concluding machine learning model being trained to identify any of the plurality of prospective target nucleic acids based on the combined input data.

The at least one unknown nucleic acid may be a plurality of unknown nucleic acids. The method may further comprise determining that each of the plurality of unknown nucleic acids is a member of the plurality of prospective nucleic acids, thereby identifying the presence of a plurality of different nucleic acids present in the solution.

According to another aspect of the present disclosure, there is a provided a computer-implemented method of training a machine learning model to identify any of a plurality of prospective target nucleic acids in a solution comprising a biological sample. The method comprises receiving amplification curve data indicative of an amplification reaction associated with at least one known nucleic acid, the known nucleic acid being one of the plurality of prospective target nucleic acids. The received data is processed, the processing comprising inputting data into a machine learning model to generate a prediction as to whether the known nucleic acid is one of the plurality of prospective target nucleic acids. The input data is based on the amplification curve data, may be indicative of the degree of amplification of the at least one known nucleic acid over time, and may be labelled according to the known nucleic acid. Based on the generated prediction, the machine learning model may be trained to identify any of the plurality of prospective target nucleic acids.

The method of training the machine learning model may further comprise receiving melting curve data associated with the at least one known nucleic acid. The melting curve data may be indicative of a degree of dissociation of the at least one known nucleic acid with increasing temperature. The input data may be further based on the melting curve data.

According to another aspect of the present disclosure, a computer readable medium is provided comprising computer executable instructions which, when performed by a processor, cause the processor to perform implementations of the disclosed methods.

### FIGURES

Specific embodiments are now described, by way of example only, with reference to the drawings, in which:

FIG. 1a depicts a typical process for nucleic acid amplification.

FIG. 1b is a graph depicting the typical profile of a negative and positive real-time amplification reaction, and in particular shows the change in pH or fluorescence over time in a DNA amplification reaction.

FIG. 2 depicts an experimental workflow according to the present disclosure;

FIGS. 3 a-f shows amplification curves and melting peaks for a number of targets;

FIG. 4 a-d depict real-time dPCR data.

FIGS. 5 a-b depict multiplexing based on final fluorescent intensity.

FIG. 6 is a visualization of the similarity between amplification curves.

FIGS. 7 a-e depict the performance of methods of the present disclosure, and in particular ACA, in the presence of single and multiple targets.

FIGS. 8 a-c depict the impact of co-amplification events within the field of digital PCR.

FIG. 9 depicts an example workflow according to the present disclosure.

FIG. 10 depicts a workflow in which melting curve data is incorporated into the ACA workflow in accordance with methods of the present disclosure.

FIG. 11 depicts a flowchart to visualise the data processing workflow.

FIG. 12 depicts how AMCA techniques may be incorporated within the ACA approach.

FIGS. 13 a-e depicts the analysis of real-time amplification and melting curves from qPCR and dPCR instruments.

FIGS. 14 a-f depict the performance of methods for multiplexing 9 mcr targets.

FIG. 15 illustrates a block diagram of one implementation of a computing device.

FIG. 16 depicts a method according to the present disclosure.

FIG. 17 depicts the beneficial effects of data augmentation.

### DETAILED DESCRIPTION

### Overview of the Present Disclosure

At the highest level, the present application relates to a method of identifying the presence of at least one target nucleic acid in a solution containing a biological sample. The method is capable of multiplexing, and as such can identify multiple different prospective target nucleic acids in solution. In overview, the method comprises receiving amplification curve data indicative of the degree of amplification of the at least one target nucleic acid with time. The amplification data may be, for example, real-time digital PCR data or real-time PCR data. This data is processed, and processing the amplification curve data comprises inputting the amplification curve data, or values derived therefrom, into a machine learning model trained to identify the presence of any of the plurality of prospective target nucleic acids. As a result, the presence of at least one target nucleic acid in the biological sample can be determined.

According to a first implementation of the present disclosure, the processing and determination is conducted on the basis of amplification curve data. Prior digital PCR approaches have used PCR reactions primarily for counting and quantifying the amount of a particular target in solution, rather than identifying which of a plurality of potential, or prospective, target nucleic acids are present in solution. Where prior approaches have used amplification curve data, they have done so by first identifying key features of the curve in order to inform a multi-dimensional analysis. While these approaches work well, the present inventors have realised that this non-trivial feature extraction step is not necessary if machine learning methods are employed. Therefore, present methods are quicker and more efficient than prior methods. To date, no prior approaches have used supervised machine learning to provide a solution to the problem of identifying which, if any, of a plurality of prospective target nucleic acids are present in a solution containing a biological sample.

According to a second implementation, the method comprises additionally receiving and processing melting curve data. The amplification curve data can be considered to provide kinetic information regarding the amplification reaction occurring in solution, and the melting curve data can be considered to provide thermodynamic information regarding the reaction occurring in solution. By inputting both melting curve and amplification curve data into a suitably trained machine learning model it is possible to improve the model's ability to multiplex still further. In other words, additionally processing melting curve data improves the method's ability to distinguish between the prospective target nucleic acids in order to improve the accuracy of the method in determining which target nucleic acid is present in solution.

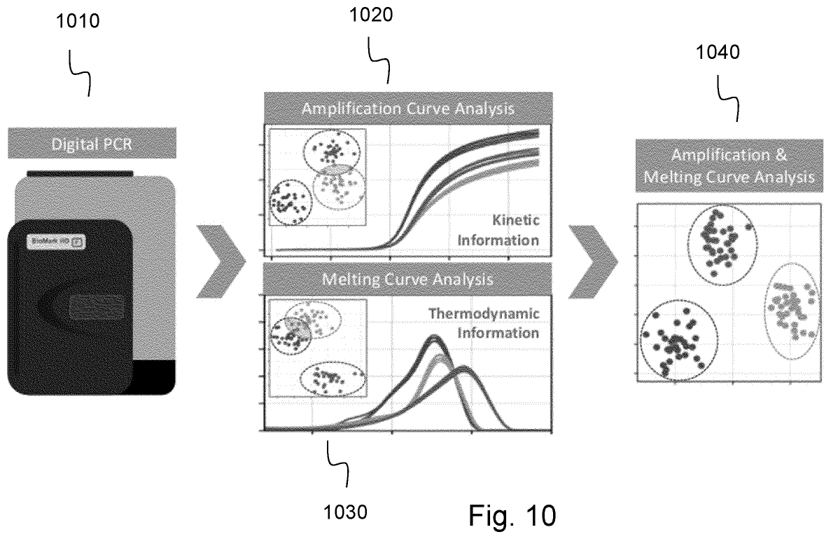

The present application will explain these two implementations in turn. The first implementation, in which the data processing is based on amplification curve data, is referred to herein as amplification curve analysis (ACA). The second implementation, in which the data processing is based on amplification curve data and melting curve data, is referred to herein as amplification and melting curve analysis (AMCA). While the methods are described primarily separately, it will be appreciated by the skilled person that the methods are highly complementary. For example, a workflow is depicted in FIG. 10 in which the melting curve data is incorporated into the ACA workflow.

Nucleic Acid Amplification

The following explanation of nucleic acid amplification relates primarily to pH based detection, and describes this detection primarily in relation to detecting DNA. This section serves to give useful background information and serve to give the reader an introduction to these concepts. However, the present disclosure is in no way limited to pH based detection, or to the detection of only DNA.

DNA amplification, the process of replicating DNA from one original DNA molecule, is used to amplify a single or a few copies of a segment of DNA generating thousands to millions of copies of a particular DNA sequence and can be used to determine whether a sample of human fluid or tissue contains DNA or RNA of a pathogen (such as viruses, bacteria, fungi or protozoa). The basic premise is that the DNA amplification is allowed if and only if the target pathogen exists. Following this, the DNA amplification is monitored. For instance, in traditional methods such as real-time polymerase chain reaction (PCR) each time a new amplicon is produced, a fluorescent molecule is released. Hence, the release of this fluorescent molecule is an indication of the presence of a pathogen in the sample.

It is also possible to monitor the pH of the chemical solution because during DNA amplification, each time a nucleotide is incorporated into the new DNA strand, Hydrogen ions are released which cause a change in the pH (pH=−log 10 [H⁺], where H⁺ is the concentration of Hydrogen ions or protons). The chemistry is summarised in the below equation where a is an integer constant.

DNA+reactants->2·DNA+α·Proton (H⁺)+products

If DNA amplification is triggered (i.e. the pathogen is present in the sample) then the reaction is defined as positive, otherwise, the reaction is described as negative.

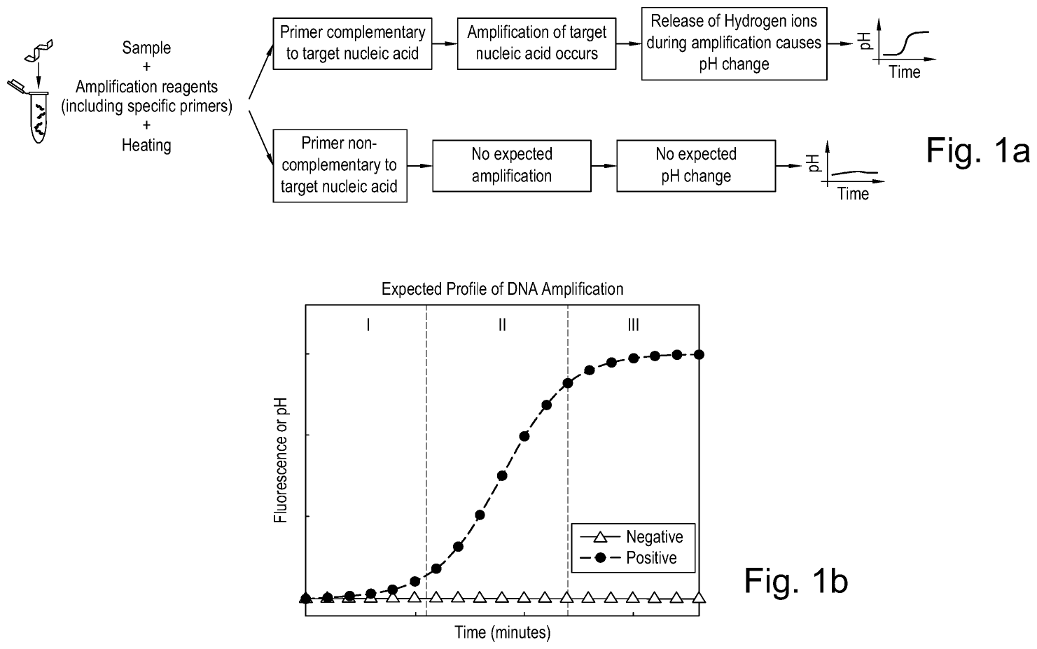

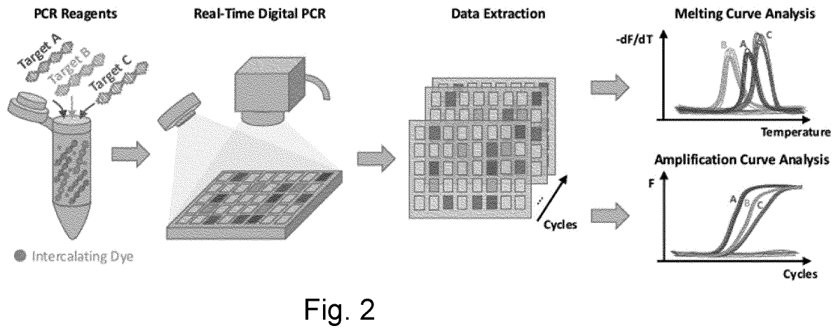

A high-level description of how pH-based DNA detection is typically performed is illustrated in FIG. 1a and summarised in the following steps:
1. Chemical solution consisting of sample and other necessary chemicals is prepared.
2. Amplification reagents associated with a specific pathogen is added to the solution. This consists of a primer, a sequence of bases, that complements the target DNA.
3. Depending on the method of DNA detection, the chemical solution may be heated.
4. Amplification is triggered if the primer complements the DNA in the sample.
5. DNA amplification is monitored, for instance, through fluorescence or pH.

Assuming no noise exists in the system, a typical output profile for DNA detection is shown in FIG. 1b. This figure includes a typical profile for a positive and a negative reaction. The graph shows time on the x-axis, and pH (or fluorescence) on the y-axis.

The graph is split into three ‘stages’ representing the expected profile for DNA amplification. At stage I) the reactants have not found each other yet. At stage II) amplification is taking place. At stage III) the reaction has saturated. The ‘time to positive’, tₚ, is defined as the time from the beginning of the reaction until a positive determination that the DNA is amplifying. Since the threshold is arbitrary, in examples used herein tₚ may be taken as the time for half of the amplification to complete.

Traditional methods of nucleic acid-based detection use optical mechanisms based on fluorescence labelling that require large and costly equipment. Typically, this equipment makes such techniques unsuitable for point-of-care diagnostics.

Polymerase chain reaction (PCR), is the most common method of nucleic acid-based detection, within which the DNA amplification is done in cycles. In each cycle, the number of DNA molecules is doubled until one of the reactants have been consumed. Each PCR cycle typically comprise three steps (denaturation, annealing and extension) and each of these steps occur at a particular temperature. PCR has an appealing property that the number of DNA molecules can be easily quantified (2^{N} where N is the number of cycles).

Digital polymerase chain reaction (dPCR) is a mature technique that has enabled scientific breakthroughs in several fields. However, this technology is primarily used in research environments with high-level multiplexing representing a major challenge. Here, we propose a novel method for multiplexing, referred to as amplification and melting curve analysis (AMCA), which leverages the kinetic information in real-time amplification data and the thermodynamic melting profile. The methods have been demonstrated using an affordable intercalating dye (EvaGreen). The method comprises training a system comprised of supervised machine learning models for accurate classification, by virtue of the large volume of data from digital PCR platforms. As an example presented herein, a new 9-plex assay is disclosed to detect mobilised colistin resistant (mcr) genes as clinically relevant targets for antimicrobial resistance. Over 100,000 amplification events have been analysed, and for the positive reactions, the AMCA approach reports a classification accuracy of 99.3%, an increase of 9.94% over using melting curve analysis. This work extends the benefits of dPCR to diagnostic pathways within clinical settings, by providing an affordable method of high-level multiplexing without fluorescent probes.

Detecting and quantifying nucleic acids are important tasks in several fields, where the real-time polymerase chain reaction (qPCR) remains the most common technique. More recently, the use of digital PCR (dPCR) has been flourishing due to the several advantages over conventional qPCR, such as: (i) lack of references or standards; (ii) high precision in quantification; (iii) tolerance to inhibitors; and (iv) the capability to analyze complex mixtures. Therefore, dPCR has enabled scientific breakthroughs in clinical microbiology, gene expression and precision cancer research, among others.

Multiplex assays provide a practical solution for nucleic acid detection in a single reaction, reducing the time, cost and amount of sample required, at the expense of technical complexity. Current approaches based on fluorescent probes are expensive and require lengthy optimisation which is not suitable for high-throughput applications. Intercalating dyes provide a suitable alternative chemistry which is affordable and does not require in-silico design. However, since intercalating dyes bind to any double-stranded DNA, the prospect of non-specific amplification is typically addressed with further post-PCR analyses such as gel electrophoresis, melting curve analysis or sequencing methods.

In multiplex dPCR, the most common approach uses the final fluorescent intensity (FFI) of the amplification curve to distinguish between targets. Reported studies show that adjusting primer concentration, the modulation of the FFI is achievable for specific target identification. However, extensive optimization is required and the number of targets is limited due to the variation of FFI values.

As described above, the new ACA method reduces the need for lengthy optimization, in part by using supervised machine learning to enable target-specific kinetic information to be extracted from real-time amplification data. However, the ability of the ACA approach to perform high level multiplexing can be improved still further by incorporating thermodynamic information extracted from the melting curve.

Some dPCR instruments offer the capability of melting curve analysis (MCA), providing a post-PCR method to identify specific targets with established literature and tools to assist assay design. However, high-level multiplexing with MCA requires non-trivial assay design to distinguish close melting curve peaks.

Although the aforementioned methods are analysing the same amplification product, they take advantage of different information to distinguish between targets. The amplification curve encodes target-specific kinetic information (i.e. complex reaction efficiency from cycle-to-cycle) while the melting curve is the result of thermodynamic properties of the amplicon (e.g. GC content and length). To date, no methods have been proposed which comprise enhancing multiplexing capabilities by combining the amplification and melting curves.

According to methods of the present disclosure, a commercially available dPCR platform (such as Fluidigm's BioMark HD) may be used with an intercalating dye (EvaGreen) to demonstrate that non-mutual information from amplification and melting curves can improve multiplexing accuracy. The proposed method, referred to as amplification and melting curve analysis (AMCA), leverages the large volume of data from real-time dPCR and trains a machine learning system. Optionally, the machine learning system is a “three-step” system.

FIG. 10 depicts the AMCA method at a very high level. At 1010, amplification and melting curve data is extracted from a real-time dPCR instrument (e.g. Fluidigm BioMark HD). In a training stage in which the amplification curve and melting curve data are representative of a known nucleic acid, the data is used to train machine learning models to classify multiple targets for both datasets individually. Subsequently, the trained models can be used to identify the presence of any of the nucleic acids which formed the basis of the training data.

At block 1020, the amplification curve data is inputted into a first machine learning model. At block 1030, the melting curve data is inputted into a second machine learning model. The ability of the machine learning models to distinguish between different target nucleic acids is visualized in the graphs. For high-level multiplexing, both methods may sometimes provide insufficient accuracy. This scenario is indicated by overlapping data distributions highlighted by the shaded regions in the graphs. However, the proposed method, referred to as amplification and melting curve analysis, or AMCA, takes into account both kinetic and thermodynamic information in order to classify the targets accurately.

At block 1020, a model is trained on the entire real-time amplification data and at block 1030 a model is trained using melting curve information. The final step, at 1040, combines the resulting outputs into a final classification for each amplification event.

The resulting classification, as visualized in the graph of block 1040, is able to distinguish between each of the nucleic acids.

As a case study, this work applies the AMCA method to the global challenge of antimicrobial resistance. In particular, colistin is a “last-line” antibiotic, reserved for the treatment of severe bacterial infections. The rise of mobilised colistin resistance (mcr) presents the possibility of untreatable infections, and has been reported in over 40 countries across five different continents.

Colistin resistant genes are often co-localised on highly transmissible plasmids and are readily shared between bacterial species, providing the ideal conditions for multi-drug resistant organisms (REF). Incorrect diagnosis delays appropriate intervention, increases financial burdens for the healthcare system and complicates antimicrobial stewardship efforts. Therefore, detecting variants of mcr is important to help treat and understand this emerging antimicrobial resistance. In this study, we develop the first 9-plex assay to detect mcr-1 to mcr-9.

By using the presently disclosed methods, in particular by using AMCA, researchers and practitioners will be able to use affordable multiplex assays, compatible with dPCR platforms, for their clinically relevant applications.

DNA Templates

Double-stranded synthetic DNA (gBlock Gene fragments) containing the entire coding sequences of mcr-1 to mcr-9 were used. The accession numbers from GenBank web site for each target are shown in Table 1. Table 1 depicts the primer sequences and relevant meta data regarding the amplicon for all nine mcr targets. All primers have been fully developed in-house and published for the first time in this study. The gBlocks were purchased from Life Technologies (ThermoFisher Scientific) and re-suspended in Tris-EDTA buffer to 10 ng/μL stock solutions (stored at −80° C. until further use). The concentrations of all DNA stock solutions were determined using a Qubit 3.0 fluorimeter (Life Technologies).

TABLE 1

| Target | Forward primer | Reverse primer | Product size |
|---|---|---|---|
| (accession number) | (5′→3′) | (5′→3′) | (bp) |
| mcr-1 (KP347127.1) | TGGCGTTCAGCAGTCATTATGC | CAAATTGCGCTTTTGGCAGCTTA | 516 |
| mcr-2 (LT598652.1) | CTGTATCGGATAACTTAGGCTTT | ATACTGACTGCTAAATAGTCCAA | 407 |
| mcr-3 (KY924928.1) | AGACACCAATCCATTTACCAGTAA | GCGATTATCATCAAACTCCTTTCT | 136 |
| mcr-4 (MF543359.1) | TTGCAGACGCCCATGGAATA | GCCGCATGAGCTAGTATCGT | 207 |
| mcr-5 (ky807921.1) | GGTTGAGCGGCTATGAAC | GAATGTTGACGTCACTACGG | 207 |
| mcr-6 (MF176240.1) | GTCCGGTCAATCCCTATCTGT | ATCACGGGATTGACATAGCTAC | 556 |
| mcr-7 (MG267386.1) | TGCTCAAGCCCTTCTTTTCGT | TTGGCGACGACTTTGGCATC | 466 |
| mcr-8 (NG061399.1) | CGAAACCGCCAGAGCACAGAATT | TCCCGGAATAACGTTGCAACAGTT | 617 |
| mcr-9 (NG_064792.1) | TATAAAGGCATTGCTTACCGTT | GGAAAGGCACTTTAGTCGTAAA | 202 |

Multiplex Primer Design

To perform the (in-silico) design for the 9-plex, an NCBI blast was conducted to ensure that each primer set binds to a conserved region. For each target, the blast was able to retrieve an average of 1000 sequences, which have been used to identify variation in the nucleotide sequence for all possible inclusive targets within the same gene and exclude potential cross-reactivity sequences (either within the mcr family or from a different species). Alignments were performed using the MUSCLE algorithm (22), in Geneious Prime® 2020.1.2. Primer characteristics were analyzed through the IDT OligoAnalyzer software using the J. SantaLucia thermodynamic table for melting temperature (Tm) evaluation, hairpin, self-dimer and cross-primer formation (24). The Tm of the amplification product of each primer set was determined by the Melting Curve Predictions Software (uMELT) package. All primers were synthesized by Life Technologies (ThermoFisher Scientific). Primer sequences are listed in Table 1.

PCR Reaction Conditions

Real-time Digital PCR. Each amplification reaction was performed in 4 μL of final volume with 2 μL of SsoFast EvaGreen Supermix with Low ROX (BioRad, UK), 0.4 μL of 20× GE Sample Loading Reagent (Fluidigm PN 85000746), 0.4 μL of 10× multiplex PCR primer mixture containing the nine primer sets (2.5 μM of each primer), and 1.2 μL of different concentrations of synthetic DNA (or controls). PCR amplifications consisted of a hot start step for 10 min at 95° C., followed by 45 cycles at 95° C. for 20 s, 66° C. for 45 s, and 72° C. for 30 s. Melting curve analysis was performed with one cycle consisting of 65° C. for 3 s and continuous reading from 65 to 97° C. with an increment of 0.5° C. every 3 s. The integrated fluidic circuit (IFC) controller was used to prime and load qdPCR 37K™ digital chips and Fluidigm's Biomark HD system to perform the dPCR experiments, following manufacturer's instructions.

Real-time PCR. Each amplification reaction was performed in 10 μL of final volume with μL of SsoFast EvaGreen Supermix with Low ROX (BioRad, UK), 3 μL of PCR grade water, 1 μL of 10× multiplex PCR primer mixture containing the nine primer sets (2.5 μM of each primer), and 1 μL of different concentrations of synthetic DNA (or controls). The reaction consisted of 10 min at 95° C., followed by 45 cycles at 95° C. for 20 s, 66° C. for 45 s, and 72° C. for 30 s. Melting curve analysis was performed with one cycle consisting of 95° C. for 10 s, 65° C. for 60 s, and 97° C. for 1 s (continuous reading from 65 to 97° C.).

Data Analysis

Multiplexing Based on FFI.

Final fluorescent intensity values were extracted from each amplification curve and used to train a logistic regression classifier to distinguish targets.

Amplification Curve Analysis, or ACA, or ACA, consists of training a supervised machine learning model to distinguish targets based on the entire real-time amplification curve.

Several different supervised learning techniques may be used. In an implementation of the present disclosure, a deep neural network was chosen based on cross-validation score. In particular, the neural architecture consists of two convolutional layers in order to extract temporal dynamics of the curve whilst keeping training times low (compared to recurrent architectures such as long short-term memory or gated recurrent unit networks). The first layer consists of 16 filters (kernel size of 5) and the second layer has 8 filters (kernel size of 3), where both layers have a rectified linear unit activation function. Prior to training the model, amplification curves were pre-processed using background subtraction (removing the mean of the first 5 fluorescent measurements) and subsequently calling positive/negative curves based on an arbitrary threshold.

Melting Curve Analysis, or MCA, consists of distinguishing the thermodynamic profile (i.e. −dF/dT) of the amplification product. In this study, and conventionally, this is achieved by distinguishing the melting peak, Tm, although methods have also been proposed to consider the entire curve (26, 27). After peak detection, negative reactions can be confirmed by identifying curves with no peak. Subsequently, a supervised machine learning model can be trained to distinguish the Tm values. In this study, logistic regression was chosen as a classifier based on cross-validation.

Method According to AMCA

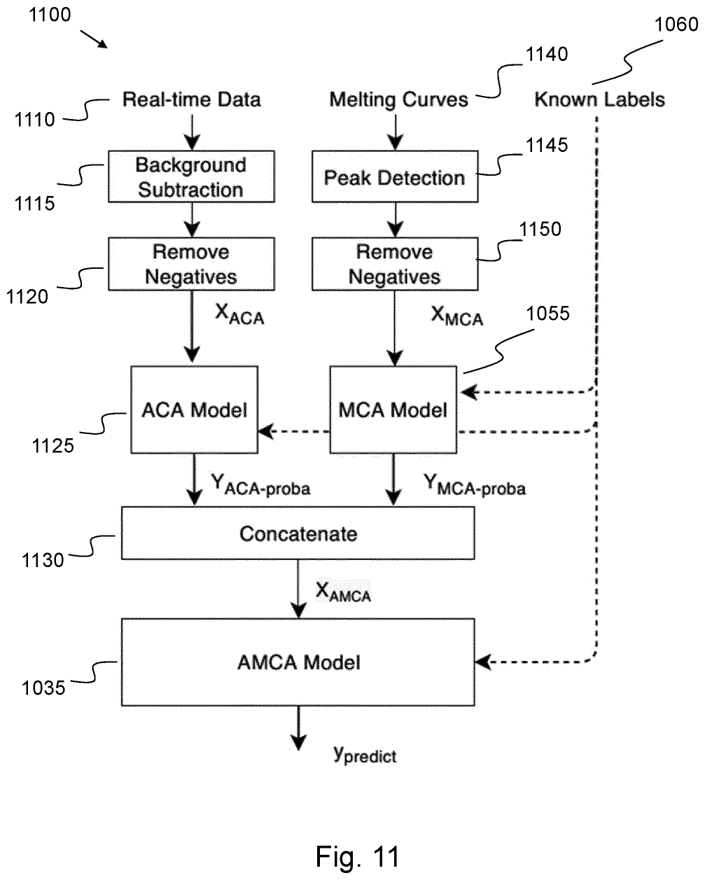

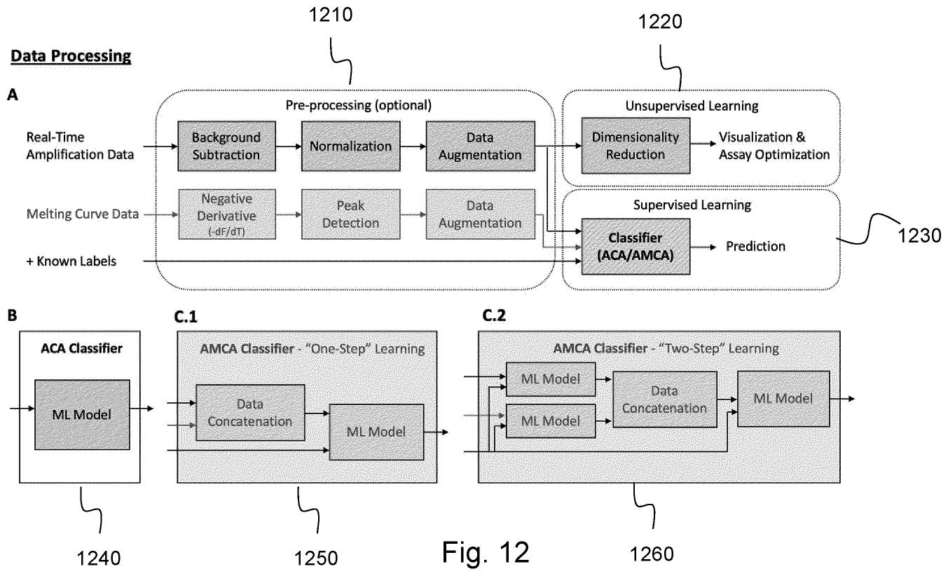

The present method, termed amplification and melting curve analysis, orAMCA, trains a supervised machine learning model to combine the predictions of ACA and MCA. This process is visualized in FIGS. 11 and 12. The output of ACA and MCA are probabilities for the amplification event belonging to each target of interest. In the training process, these probabilities are concatenated and used to train a model. In this study, a logistic regression classifier was chosen. It is important to note that this classifier is tuned with its own cross-validation step in order to avoid over-fitting.

FIG. 11 depicts a flowchart to visualise the data processing workflow 1100 for the presently disclosed method. Known labels 1060 (marked with a dashed line) are only required for training the models, as opposed to testing unknown samples. The workflow will be discussed primarily with respect to the testing of unknown samples. At step 1110, real-time amplification curve data is received. This data may be indicative of an amplification reaction associated with at least one unknown nucleic acid present in the solution. At steps 1115 and 1120, pre-processing is performed. In particular, the background is subtracted from the data and negatives are removed. In other words, negative amplification events, i.e. no target nucleic acid present in the solution, is not used to train the ML model. The result is pre-processed amplification curve data, XACA, which is indicative of the degree of amplification of an unknown nucleic acid in solution over time.

The pre-processed amplification curve data is inputted into a trained classifier at block 1125. The trained classifier mis a first machine learning model, which may be referred to as an ACA model or a trained ACA model. The output of the first machine learning model is a prediction, Y_{ACA-proba} for the amplification event represented by the amplification curve data being caused by one of a plurality of prospective target nucleic acids.

T block 1140, melting curve data is received. The melting curve data is indicative of the degree of dissociation of the unknown nucleic acid in solution. At 1145, and 1150, the data is pre-processed. At 1145, the melting curve peak is detected. Peaks may be detected in any of several different known ways. Peak detection is a common activity in signal processing and the skilled person will be familiar with methods of peak detection. At 1150, negatives are removed. The result of the pre-processing steps is pre-processed melting curve data X_{MCA-proba}. This data is inputted into a trained classifier at block 1055. The trained classifier is a second machine learning model, which may be referred to as an MCA model or a trained MCA model. The output of the second machine learning model is a prediction, Y_{MCA-proba} for the amplification event represented by the melting curve data being caused by one of a plurality of prospective target nucleic acids.

At block 1130, the outputs from each of the first and second machine learning models, i.e. the ACA and MCA models, are concatenated such that the concatenated output, X_{AMCA}, may be inputted into a third machine learning model, which may be referred to as an AMCA model or a trained AMCA model. The output of this model is a prediction, y_{predict}, of which target nucleic acid of the prospective target nucleic acids is present in solution, i.e. which nucleic acid caused the amplification event represented by the amplification and melting curve data.

Each of the first, second and third machine learning models are trained using known methods using the known labels 1060, which are obtained via extracting amplification and melting curve data from reactions containing the target nucleic acids. Together, the first, second and third machine learning models may be referred to as a machine learning system.

FIG. 12 depicts a similar workflow to that show in FIG. 11, but indicates more clearly how AMCA techniques may be incorporated within the ACA approach. At block 1210, received amplification curve data is pre-processed. Optionally, received melting curve data is also pre-processed. The pre-processing block generates input data which is suitable for inputting into a machine learning model, or models. Alternatively, there may be no pre-processing stage, in which case the input data may simply be the received amplification curve and melting curve data.

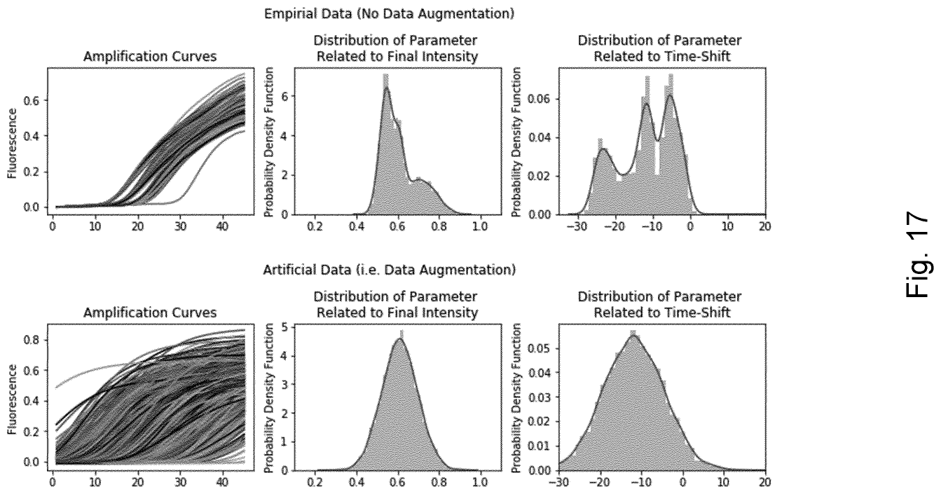

Re-processing may further comprise data augmentation, as will be described below in relation to FIG. 17.

The amplification curve input data may be passed to an unsupervised model at block 1220 to assist with visualizing the distinguishability of the various targets.

The received data is processed at block 1230. Processing the received data comprises inputting the input data into a machine learning model, e.g. a classifier, trained to identify any of the plurality of prospective target nucleic acids. For an ACA method, the classifier is an ACA classifier capable of generating a determination that an unknown nucleic acid in solution, represented by the received amplification curve data, is one of a plurality of prospective nucleic acids which the classifier has been trained to identify.

In an AMCA approach, melting curve data is incorporated into this workflow in the manner depicted. In this case, the input data which is inputted into the machine learning model at block 1230 is combined input data, which is based on both the received melting curve data and the received amplification curve data.

According to the approach used, block 1230 can be represented by any of blocks 1240, 1250, or 1260.

As will be appreciated from block 1260, in some implementations the method may comprise a two-step machine learning system. The method therefore may comprise inputting first input data into the first machine learning model, the first input data being based on the received amplification curve data and the first machine learning model being trained to identify any of the plurality of prospective target nucleic acids based on the first input data; inputting second input data into the second machine learning model, the second input data being based on the received melting curve data and the second machine learning model being trained to identify any of the plurality of prospective target nucleic acids based on the second input data; generating the combined input data based on outputs from the first and second machine learning models; and inputting the combined input data into the concluding machine learning model, the concluding machine learning model being trained to identify any of the plurality of prospective target nucleic acids based on the combined input data. The combined data may be generated by concatenating the results of the first and second machine learning model in the manner shown in block 1260.
1. Pre-processing (optional)
   For real-time amplification data, methods include but not limited to background subtraction, normalization, sigmoidal fitting and data augmentation (i.e. artificially increasing the training data set).
      Used in ACA and AMCA
   For melting curve data, methods include but not limited to taking the negative derivative, performing peak detection and data augmentation.
      Used in AMCA
2. Unsupervised learning
   Dimensionality reduction techniques can be used to visualize the similarity between data points and support the optimization of the multiplex assay. Examples include, but not limited to, t-SNE and PCA.
      Used in ACA and AMCA
3. Supervised learning
   ACA (Data Processing B)—The input to the ‘classifier’ is the entire real-time amplification curve after pre-processing. Examples include but not limited to k-nearest neighbours, support vector machines and deep neural networks.
   AMCA (Data Processing C.1 or C.2)—The input to the ‘classifier’ is the entire real-time amplification curve and melting curve after pre-processing. There are two approaches in implementing the classifier which includes machine learning models (e.g. including but limited to k-nearest neighbours, support vector machines and deep neural networks).
      “One-step learning” (C.1)—The amplification and melting curves are concatenated and fed into a single supervised learning model.
      “Two-step learning” (C.2)—First, two models are trained, one for amplification data and one for melting curve data. Subsequently, the output of these models are concatenated and used to train another model. Note: Each model can use different machine learning algorithms.

Data Augmentation

The pre-processed data can be optionally passed into a ‘data augmentation’ process to artificially increase the volume of data in order to improve the classification performance. For example, to account for the variation in the final fluorescent intensity or time-shift (i.e. concentration of initial nucleic acids) of the amplification curves, a sigmoid model can be fit to the amplification curves. Subsequently, a distribution (e.g. normal or uniform or non-parametric) can be fit to the parameters of the model related to the final fluorescent intensity or time-shift, and via sampling, ‘new’ curves can be generated. This is visualized in FIG. 17, where the top panels illustrates real-world data, and the bottom panels shows the curves after data augmentation. Similar data augmentation techniques may be used for melting curve data.

Statistical Analysis

Performance of the models was evaluated based on out-of-sample classification accuracy, as determined by 10-fold cross-validation (using stratified splits). In order to assess the performance as a function of the volume of training data, a shuffled stratified split was performed 10 times, with 5000 test samples. The two-sided t-test with unknown but unequal variances was used to determine statistical significance for comparing the classification accuracy of different models. Prior to this test, a Kolmogorov-Smirnoff test was used to determine normality of the distributions and an F-test for equal/unequal variances. A p-value of 0.05 was used as a threshold for statistical significance for all tests used in this study.

A New Multiplex Assay for Mobilised Colistin Resistance

To date, there has been no report of multiplexing all mcr variants together. Here, a new 9-plex has been designed using a conventional qPCR platform.

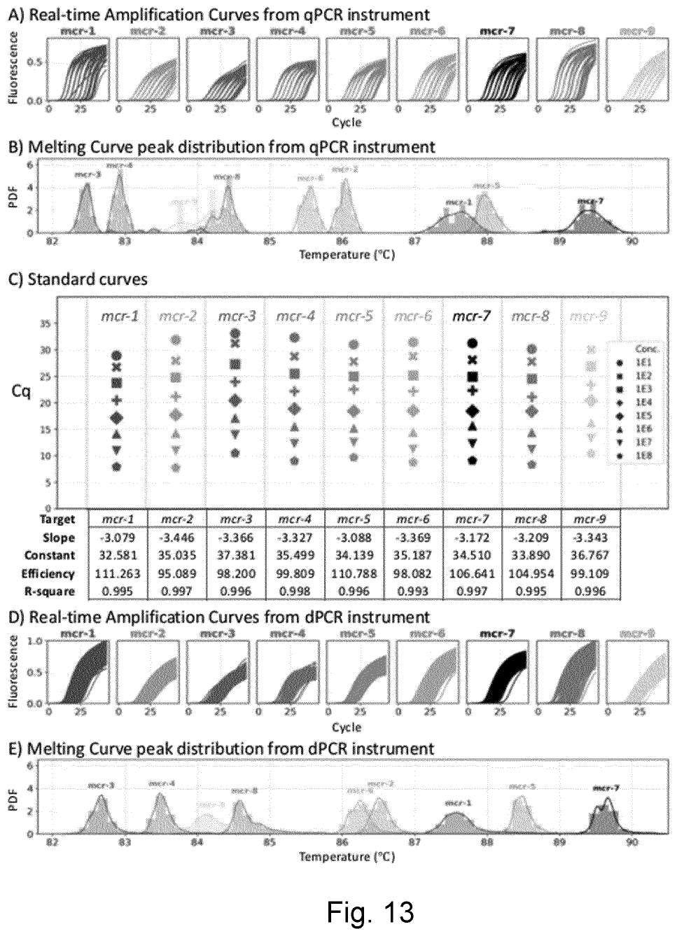

FIG. 13 depicts the analysis of real-time amplification and melting curve from qPCR and dPCR instruments. A) Real-time amplification curves from qPCR instrument. B) Melting curve peak distribution from qPCR instrument showing the probability density function (PDF) for each target. The mean std of mcr-1 to mcr-9 is 87:6 0:2 C, 86:0 0:1 C, 82:6 0:4 C, 82:9 0:1 C, 88:0 0:1 C, 85:5 0:1 C, 89:4 0:2 C, 84:4 0:1 C, 84:1 0:2 C, respectively. C) Visualisation and statistics of standard curves for a serial dilution of each target in qPCR using 9-plex assay. D) Real-time amplification curves from dPCR instrument. E) Melting curve peak distribution from dPCR instrument. The mean std of mcr-1 to mcr-9 is 87:7 0:3 C, 86:6 0:2 C, 82:7 0:2 C, 83:6 0:2 C, 88:5 0:2 C, 86:3 0:2 C, 89:7 0:2 C, 84:8 0:3 C, 84:3 0:3 C, respectively.

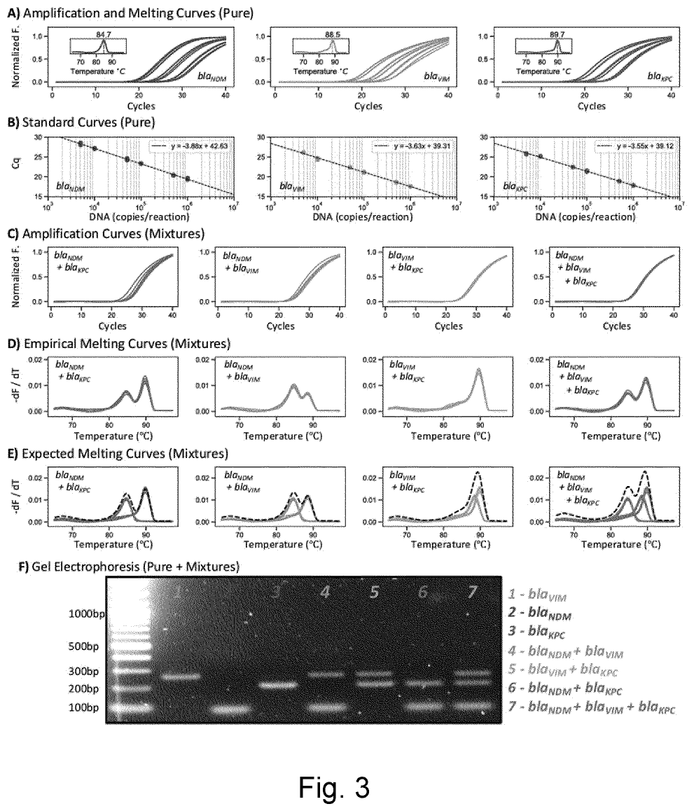

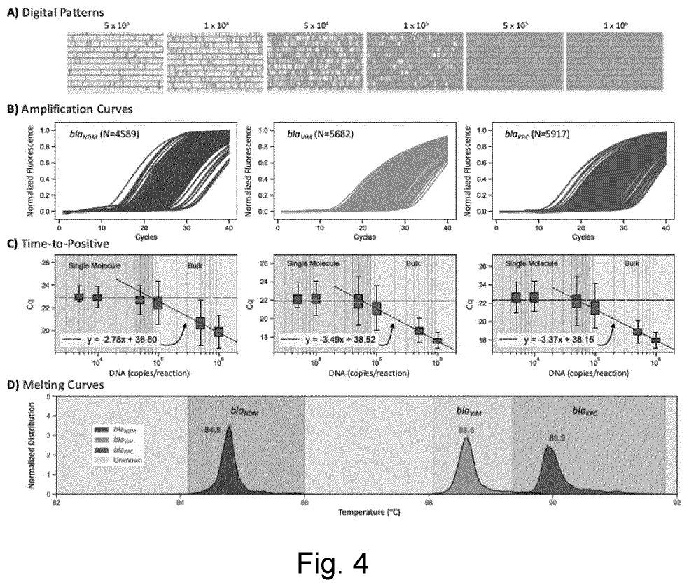

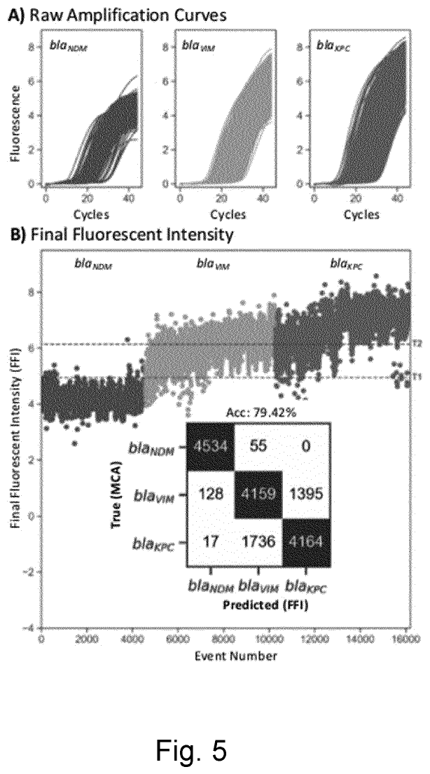

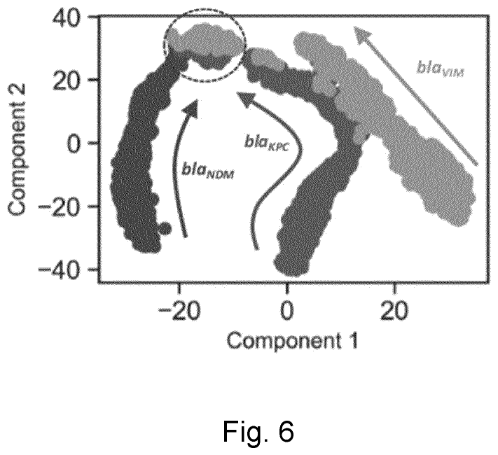

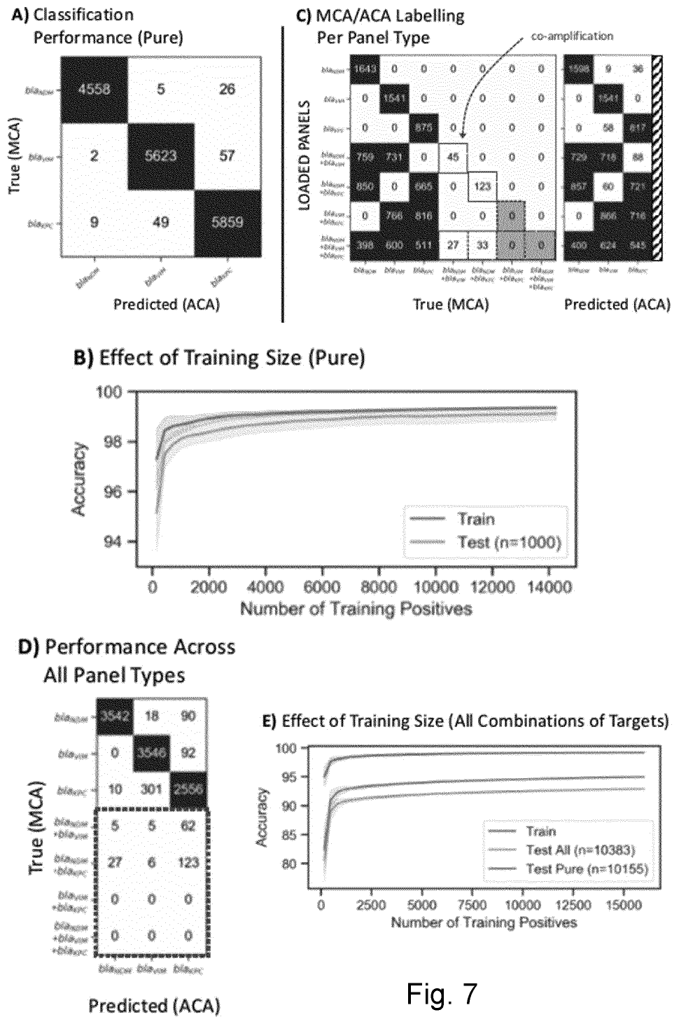

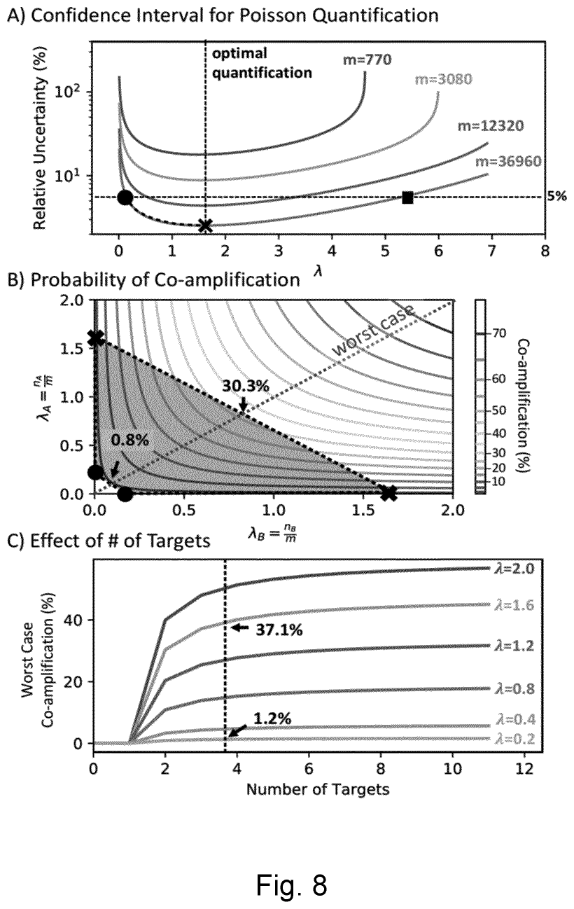

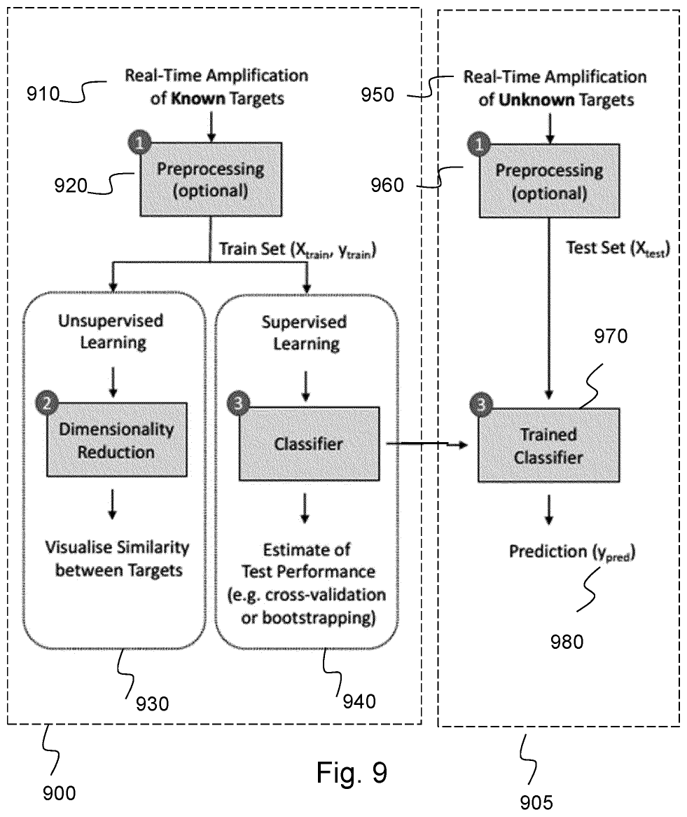

FIGS. 13 (A)-(C) show the real-time amplification curves, melting peak distributions and standard curves for a serial dilution of each target. It can be observed that the distribution of FFI values and the shape of each target is different, although the precise overlap cannot be visualised since the curves are in 45-dimensional space. On the other hand, the melting peak distributions have distinct mean Tm values, although some targets (e.g. mcr-1 and mcr-5) have overlapping distributions, compromising MCA multiplexing classification. FIG. 3 (C) demonstrates that the multiplex assay is highly efficient (all >95%) with a lower limit of detection (LoD) down to 10 copies per reaction for all targets (excluding mcr-9 which showed an LoD of 100 copies per reaction). All negative controls did not amplify before 45 cycles. The data suggests that the co-presence of mcr variants, by virtue of the overlapping Tm distributions, raise the possibility of ‘merging peaks’, demonstrating the advantage of multiplexing in digital PCR due to single-molecule partitioning.

Performance of FFI, ACA and MCA in dPCR is Limited

To assess the performance of previously reported methods, 110,880 amplification events were analysed, of which 58,664 are considered positive. FIGS. 13 (D) and (E) show the amplification and melting curves resulting from the dPCR platform, respectively. It is interesting to observe that the amplification curves and melting peak distributions resemble the qPCR data, highlight the consistency and reproducibility of the PCR chemistry and multiplex assay.

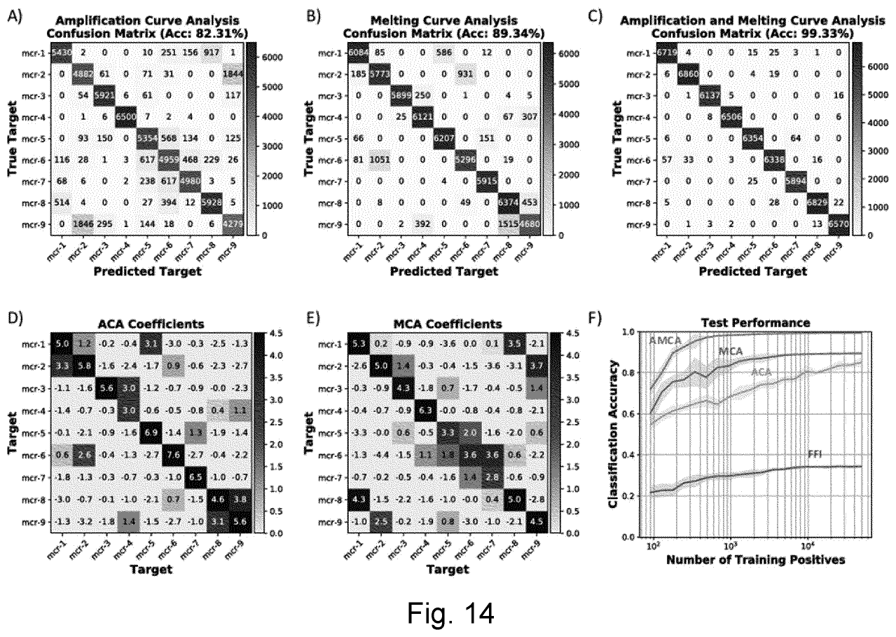

FIG. 14 depicts the performance of all methods for multiplexing the 9 mcr targets. A, B, C) The confusion matrix illustrating the predictions from ACA, MCA and AMCA (proposed method), respectively. Values indicate the number of amplification events with diagonal entries corresponding to correct predictions. D, E) Coefficients of the AMCA model weighting the predictions from the ACA and MCA methods, respectively. F) The effect of the number of training data points on the overall classification accuracy for all methods. The shaded regions correspond to 1 standard deviation.

FIG. 14 (A) shows the confusion matrices, comparing the true and predicted targets for FFI, ACA and MCA, and the overall classification performance is 25.60%, 66.69% and 84.17%, respectively. As the results indicate, the FFI performance has low accuracy due to single-parameter usage, which contains little information specific to each target. Therefore, extensive optimization for primer concentration must be performed to achieve acceptable classification accuracy, although this is neither trivial nor guaranteed. On the other hand, analysing the entire amplification curves (without normalizing for FFI) using a neural network boosts performance by 40%, extracting relevant kinetic information from each event. The third method, MCA, analysed thermodynamic information encoded in the melting profiles, showing a further increase of 15% in classification accuracy. It is interesting to observe that there is no obvious mis-classification which is evident in both ACA and MCA, suggesting that the two methods extract non-mutual information.

The AMCA method Increases Classification Accuracy Beyond 99% FIG. 14 (C) shows the confusion matrix comparing the predicted classification from the proposed method to the true labels. It can be observed that the accuracy is 99.28% and that no target is misclassified more than 2.5%. Since the chosen supervised machine learning model for AMCA is linear, the coefficients can be investigated to understand how it weighs the predictions from ACA and MCA. More specifically, the output of AMCA is defined by:

y=Ŵ_{ACA}y_{ACA}+Ŵ_{MCA}y_{MCA}

Where y_{ACA}ϵ [symbol] ⁹ and y_{MCA}ϵ [symbol] ⁹ are the probability vectors outputted from the ACA and MCA models, Ŵ_{ACA}ε [symbol] ^{9λ9} and Ŵ_{MCA}∈ [symbol] ^{9×9} are the model coefficients, respectively. FIGS. 14 (D) and (E) show the ACA and MCA coefficients in form of a heatmap, respectively. It is interesting to observe that AMCA weighs the prediction from ACA more heavily for targets which show poor classification in MCA, and vice-versa. For example, MCA misclassifies mcr-9 as mcr-8, therefore the AMCA positively weighs the ACA prediction and negatively weights the MCA prediction. Similarly, ACA misclassifies mcr-9 as mcr-2 and the coefficients compensate for this phenomenon.

The Effect of the Volume of Training Data

From a practical perspective, it is important to understand the volume of data for training the AMCA model, denoted by ₜᵣₐᵢₙ, for accurate classification. FIG. 14 (F) shows the classification performance on 5000 out-of-sample data points (repeated 10 times) where nₜᵣₐᵢₙ∈[1.0×10², 5.4×10⁴] for all models. It can be observed that all of the models perform better given more training data points. Since AMCA weighs ACA and MCA, it is unlikely to perform worse than either of it's constituents. In fact, the AMCA model consistently outperforms the others for all training data sizes and repeats. This observation is non-trivial and demonstrates that combining the kinetic information and thermodynamic profile contains more information specific to each target, enhancing multiplexing capabilities.

AMCA Method can be Translated to Conventional Real-Time PCR Platform

It is natural to ask whether the AMCA method can be translated to conventional qPCR instrument, given that machine learning benefits from sufficient volume of data. The same methodology (as in FIG. 12) was applied to the qPCR data presented in FIGS. 13 (A) and (B). The classification accuracy for FFI, ACA, MCA and AMCA was shows to be X %, Y %, Z % and A %, respectively. The confusion matrices for each method and the model coefficients for AMCA are provided in FIGS. S1 and S2. These results suggest that the AMCA method works across real-time platforms, both quantitative and digital, although a further study (outside the scope of this manuscript) is required.

Summary and Advantages of AMCA

AMCA methods enhance the capability of high-level multiplexing in real-time digital PCR platforms, increasing the classification accuracy by combining kinetic and thermodynamic information. Even a non-ideal multiplex based on ACA or MCA may in fact contain sufficient information when combined together to perform high-level multiplexing, reducing the need for further time and resource consuming optimisation.

Since in some implementations of AMCA three different models are trained, this may take time and expertise in data science to perform, especially if neural network models are used. However, computational resources have negligible cost given the wide open-source tools available for machine learning.

The ACA approach experiences a phenomenon called ‘co-amplification’, which refers to the co-presence of multiple targets in a single chamber in dPCR instruments. This problem can be solved by keeping the occupancy of the digital panel (using Poisson statistics) within acceptable bounds in order to simultaneously reduce co-amplification and retain sufficient quantification precision. For example, in the above-described 9-plex for mcr, the present inventors do not expect the co-presence of more than 2 mcr variants in the same sample, therefore. under the constraint of 36960 chambers (Fluidigm® 37K chip), the quantification uncertainty is below 5% between 16.7% and 99.3% digital occupancy.

In summary, a new method for high-multiplexing is disclosed, preferably in real-time digital PCR instruments with melting curve capabilities. This approach is based on training supervised machine learning algorithms to extract kinetic and thermodynamic information together, to enhance the classification accuracy in multiplexing. A 99.3% accuracy has been shown for identifying 9 clinically relevant targets, namely mobilised colistin resistance, using a new multiplex assay based on an affordable intercalating dye. The method may be used with conventional qPCR instruments, isothermal chemistries and electrochemical sensing technologies. And will be extremely beneficial for the wider scientific community in these areas.

It will be understood that the above description of specific embodiments is by way of example only and is not intended to limit the scope of the present disclosure. Many modifications of the described embodiments are envisaged and intended to be within the scope of the present disclosure. The following disclosure is relevant to each of the methods and approaches disclosed herein, and in particular is relevant to both ACA and AMCA.

### SUMMARY OF DISCLOSED METHODS

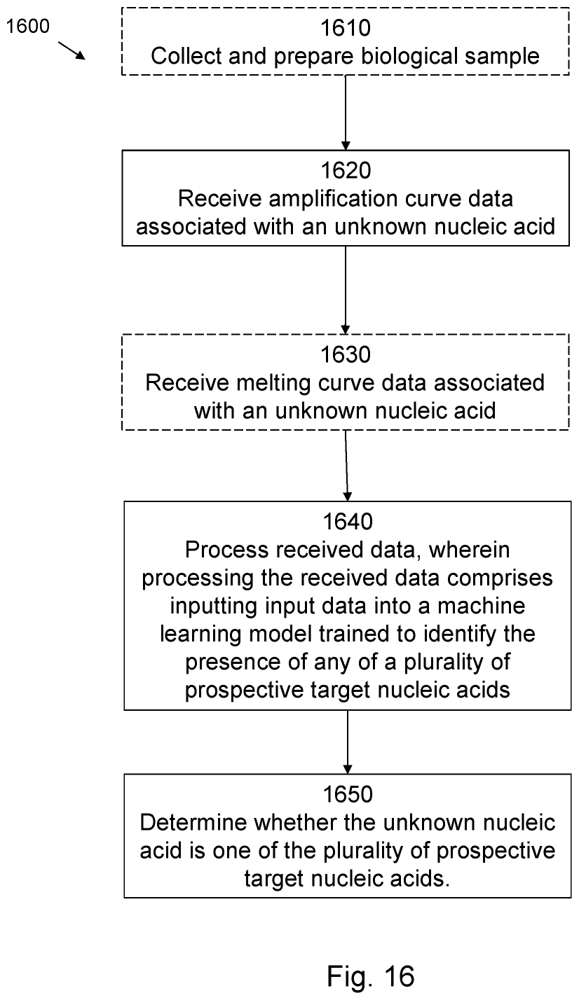

FIG. 16 is a flowchart depicting a method in accordance with the present disclosure. FIG. 16 acts as a summary of disclosed methods. Dashed lines depict optional steps in the flowchart.

At 1610, a biological sample is collected and prepared. At the highest level, this stage involves placing a biological sample in solution.

At 1620, amplification curve data is received. The amplification curve data may be received from a thermocycler or a device configured to perform an amplification reaction. The amplification curve data is indicative of an amplification reaction associated with at least one unknown nucleic acid present in the solution. The amplification curve data is indicative of the degree of amplification of the at least one unknown nucleic acid over time during the amplification reaction.

The amplification curve data and/or the input data may comprise a time series depicting the degree of amplification over time throughout a majority of the duration of the amplification reaction.

Optionally, at step 1630, melting curve data is received. The melting curve data is also associated with the at least one unknown nucleic acid. The melting curve data is indicative of a degree of dissociation of the at least one unknown nucleic acid with increasing temperature in solution, or even for the entirety of the duration of the amplification reaction. The entirety of the reaction can be understood to be from an initial phase in which no amplification is occurring until at least a saturation phase.

At step 1640, the received data is processed. The input data is based on the data received at step 1620 and, optionally, may be further based on the data received at step 1630. The processing comprises inputting the input data into a machine learning model trained to identify any of the plurality of prospective target nucleic acids, wherein the input data is based on the amplification curve data and, like the received amplification curve data, is indicative of the degree of amplification of the at least one unknown nucleic acid over time during the amplification reaction.

Though not shown in the flowchart, the method may further comprise pre-processing the amplification curve data to generate the input data, wherein pre-processing comprises any of background subtraction and normalization. Regardless of whether pre-processing techniques are used, and if so which pre-processing techniques are used, the data inputted into the machine learning model is indicative of the degree of amplification of the at least one unknown nucleic acid over time during the amplification reaction.

At step 1650, it is determined whether the unknown nucleic acid is one of the plurality of prospective target nucleic acids. Based on the processing at block 1640, determining that the at least one unknown nucleic acid is one of the plurality of prospective nucleic acids, and thereby identifying the presence of at least one of the plurality of target nucleic acids in the solution. Thereby, the unknown nucleic acid in solution is identified.

Blocks 1620-1640 may be performed in real-time as the amplification reaction is ongoing. Data may be continually received by a processor at blocks 1620 and 1630, and continuously fed into the machine learning model as input data at 1640.

### The Biological Sample and Solution

The sample may be any suitable sample comprising a nucleic acid. For example, the sample may be an environmental sample or a clinical sample. The sample may also be a sample of synthetic DNA (such as gBlocks) or a sample of a plasmid. The plasmid may include a gene or gene fragment of interest.

The environmental sample may be a sample from air, water, animal matter, plant matter or a surface. An environmental sample from water may be salt water, waste water, brackish water or fresh water. For example, an environmental sample from salt water may be from an ocean, sea or salt marsh. An environmental sample from brackish water may be from an estuary. An environmental sample from fresh water may be from a natural source such as a puddle, pond, stream, river, lake. An environmental sample from fresh water may also be from a man-made source such as a water supply system, a storage tank, a canal or a reservoir. An environmental sample from animal matter may, for example, be from a dead animal or a biopsy of a live animal. An environmental sample from plant matter may, for example, be from a foodstock, a plant bulb or a plant seed. An environmental sample from a surface may be from an indoor or an outdoor surface. For example, the outdoor surface be soil or compost. The indoor surface may, for example, be from a hospital, such as an operating theatre or surgical equipment, or from a dwelling, such as a food preparation area, food preparation equipment or utensils. The environmental sample may contain or be suspected of containing a pathogen. Accordingly, the nucleic acid may be a nucleic acid from the pathogen.

The clinical sample may be a sample from a patient. The nucleic acid may be a nucleic acid from the patient. The clinical sample may be a sample from a bodily fluid. The clinical sample may be from blood, serum, lymph, urine, faeces, semen, sweat, tears, amniotic fluid, wound exudate or any other bodily fluid or secretion in a state of heath or disease. The clinical sample may be a sample of cells or a cellular sample. The clinical sample may comprise cells. The clinical sample may be a tissue sample. The clinical sample may be a biopsy.

The clinical sample may be from a tumour. The clinical sample may comprise cancer cells. Accordingly, the nucleic acid may be a nucleic acid from a cancer cell.

The sample may be obtained by any suitable method. Accordingly, the method of the invention may comprise a step of obtaining the sample. For example, the environmental air sample may be obtained by impingement in liquids, impaction on solid surfaces, sedimentation, filtration, centrifugation, electrostatic precipitation, or thermal precipitation. The water sample may be obtained by containment, by using pour plates, spread plates or membrane filtration. The surface sample may be obtained by a sample/rinse method, by direct immersion, by containment, or by replicate organism direct agar contact (RODAC).

The sample from a patient may contain or be suspected of containing a pathogen. Accordingly, the nucleic acid may be a nucleic acid from the pathogen. Alternatively, the nucleic acid may be a nucleic acid from the host.

The method of the invention may be an in vitro method or an ex vivo method.

The pathogen may be a eukaryote, a prokaryote or a virus. The pathogen may be found in or from an animal, a plant, a fungus, a protozoan, a chromist, a bacterium or an archaeum.

As used herein, “nucleic acid sequence” may refer to either a double stranded or to a single stranded nucleic acid molecule. The nucleic acid sequence may therefore alternatively be defined as a nucleic acid molecule. The nucleic acid molecule comprises two or more nucleotides. The nucleic acid sequence may be synthetic. The nucleic acid sequence may refer to a nucleic acid sequence that was present in the sample on collection. Alternatively, the nucleic acid sequence may be an amplified nucleic acid sequence or an intermediate in the amplification of a nucleic acid sequence.

As used herein, “anneal”, “annealing”, “hybridise” and “hybridising” refer to complementary sequences of single-stranded regions of a nucleic acid pairing via hydrogen bonds to form a double-stranded polynucleotide. As used herein, “anneal”, “anneals”, “hybridise” and “hybridises” may refer to an active step. Alternatively, as used herein, “anneal”, “anneals”, “hybridise” and “hybridises” may refer to a capacity to anneal or hybridise; for example, that a primer is configured to anneal or hybridise and/or that the primer is complementary to a target. Accordingly, for example, a reference to a primer or a region of a primer which anneals to a nucleic acid sequence or a region of a nucleic acid sequence may in a method of the invention mean either that the annealing is a required step of the method; that the primer or region of the primer is complementary to the nucleic acid sequence or region of the nucleic acid sequence; or that the primer or region of the primer is configured to anneal to the nucleic acid sequence or region of the nucleic acid sequence.

The term “primer” as used herein refers to a nucleic acid, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e. in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the nucleic acid primer typically contains 15 to 25 or more nucleotides, although it may contain fewer or more nucleotides. According to the present invention a nucleic acid primer typically contains 13 to 30 or more nucleotides.

The nucleic acid may be isolated, extracted and/or purified from the sample prior to use in the method of the invention. The isolation, extraction and/or purification may be performed by any suitable technique. For example, the nucleic acid isolation, extraction and/or purification may be performed using a nucleic acid isolation kit, a nucleic acid extraction kit or a nucleic acid purification kit, respectively.

The method of the invention may further comprise an initial step of isolating, extracting and/or purifying the nucleic acid from the sample. The method may therefore further comprise isolating the nucleic acid from the sample. The method may further comprise extracting the nucleic acid from the sample. The method may further comprise purifying the nucleic acid from the sample. Alternatively, the method may comprise direct amplification from the sample without an initial step of isolating, extracting and/or purifying the nucleic acid from the sample. Accordingly, the method may comprise lysing cells in the sample or amplifying free circulating DNA.

Following isolation, extraction and/or purification, the nucleic acid may be used immediately or may be stored under suitable conditions prior to use. Accordingly, the method of the invention may further comprise a step of storing the nucleic acid after the extracting step and before the amplifying step.

The step of obtaining the sample and/or the step of isolating, extracting and/or purifying the nucleic acid from the sample may occur in a different location to the subsequent steps of the method. Accordingly, the method may further comprise a step of transporting the sample and/or transporting the nucleic acid.

The method may further comprise diagnosing a pathogen, an infectious disease, antimicrobial resistance or a drug resistant infection if the nucleic acid molecule is present.

In particular, antimicrobial resistance may involve the spread of bacteria that produce enzymes that inactivate the widely used carbapenem antibiotics, which may be known as carbapenemase-producing organisms (CPO). More specifically, major carbapenem-resistant genes can be targeted i.e. beta-lactamase, such as blaVIM, blaOXA-48, blaNDM, blaIMP and blaKPC. Identifying these genes would improve patient outcomes and prevent the spread of antimicrobial resistance. Accordingly, the computer implemented method of identifying target nucleic acids may comprise identifying these genes.

The method of diagnosis may be an in vitro method or an ex vivo method.

The infectious disease may be selected from the group consisting of Adenovirus, Coronavirus, Human Rhinovirus, Human Metapneumovirus, Parainfluenza, Respiratory Syncytial Virus, Bordetella Acute Flaccid Myelitis (AFM), Anaplasmosis, Anthrax, Babesiosis, Botulism, Brucellosis, Burkholderia mallei (Glanders), Burkholderia pseudomallei (Melioidosis), Campylobacteriosis (Campylobacter), Carbapenem-resistant Infection (CRE/CRPA), Chancroid, Chikungunya Virus Infection (Chikungunya), Chlamydia, Ciguatera, Clostridium difficile Infection, Clostridium perfringens (Epsilon Toxin), Coccidioidomycosis fungal infection (Valley fever), Creutzfeldt-Jacob Disease, transmissible spongiform encephalopathy (CJD), Cryptosporidiosis (Crypto), Cyclosporiasis, Dengue, 1,2,3,4 (Dengue Fever), Diphtheria, E. coli infection (E. coli), Eastern Equine Encephalitis (EEE), Ebola, Hemorrhagic Fever (Ebola), Ehrlichiosis, Encephalitis, Arboviral or parainfectious, Enterovirus Infection, Non-Polio (Non-Polio Enterovirus), Enterovirus Infection, D68 (EV-D68), Giardiasis (Giardia), Gonococcal Infection (Gonorrhea), Granuloma inguinale, Haemophilus influenza disease, Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster, zoster VZV (Shingles), Histoplasmosis infection (Histoplasmosis), Human Immunodeficiency Virus/AIDS (HIV/AIDS), Human Papillomarivus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LVG), Malaria, Measles, Meningitis, Viral (Meningitis, viral), Meningococcal Disease, Bacterial (Meningitis, bacterial), Middle East Respiratory Syndrome Coronavirus (MERS-CoV), Mumps, Norovirus, Paralytic Shellfish Poisoning (Paralytic Shellfish Poisoning, Ciguatera), Pediculosis (Lice, Head and Body Lice), Pelvic Inflammatory Disease (PID), Pertussis (Whooping Cough), Plague, Bubonic, Septicemic, Pneumonic (Plague), Pneumococcal Disease (Pneumonia), Poliomyelitis (Polio), Powassan, Psittacosis, Pthiriasis (Crabs, Pubic Lice Infestation), Pustular Rash diseases (Small pox, monkeypox, cowpox), Q-Fever, Rabies, Ricin Poisoning, Rickettsiosis (Rocky Mountain Spotted Fever), Rubella, Including congenital (German Measles), Salmonellosis gastroenteritis (Salmonella), Scabies Infestation (Scabies), Scombroid, Severe Acute Respiratory Syndrome (SARS), Shigellosis gastroenteritis (Shigella), Smallpox, Staphyloccal Infection, Methicillin-resistant (MRSA), Staphylococcal Food Poisoning, Enterotoxin-B Poisoning (Staph Food Poisoning), Staphylococcal Infection, Vancomycin Intermediate (VISA), Staphylococcal Infection, Vancomycin Resistant (VRSA), Streptococcal Disease, Group A (invasive) (Strep A), Streptococcal Disease, Group B (Strep-B), Streptococcal Toxic-Shock Syndrome, STSS, Toxic Shock (STSS, TSS), Syphilis, primary, secondary, early latent, late latent, congenital, Tetanus Infection, tetani (Lock Jaw), Trichonosis Infection (Trichinosis), Tuberculosis (TB), Tuberculosis (Latent) (LTBI), Tularemia (Rabbit fever), Typhoid Fever, Group D, Typhus, Vaginosis, bacterial (Yeast Infection), Varicella (Chickenpox), Vibrio cholerae (Cholera), Vibriosis (Vibrio), Viral Hemorrhagic Fever (Ebola, Lassa, Marburg), West Nile Virus, Yellow Fever, Yersenia (Yersinia), Zika Virus Infection (Zika) and COVID-19.

The skilled person will be familiar with many amplification chemistries, and this disclosure is not limited to any particular chemistry or reaction. Similarly, the disclosure is not limited to any particular amplification instrument. Suitable amplification instruments include any instrument capable of real-time measurements including bulk (such as qPCR platform) or single-molecule (such as dPCR platform). The method can be used with single-channel or multi-channel instruments. For example, an instrument with 5 channels (i.e. each channel reads a different colour), may be used, in which 3 targets are multiplexed per channel, totaling 15 targets in a single reaction. Similarly, the present disclosure is not limited to any particular sensing method. Sensing methods may be (i) Fluorescent based, including probe-based (e.g. Taqman, Scorpion, FRET) or dye-based (e.g. SYBR, EvaGreen, SYTO). (ii) Colorimetric based. (iii) Electrochemical based (e.g. pH or ion based sensing).

A Computing Device and a Computer Readable Medium

The approaches described herein may be embodied on a computer-readable medium, which may be a non-transitory computer-readable medium. The computer-readable medium carrying computer-readable instructions arranged for execution upon a processor so as to make the processor carry out any or all of the methods described herein.

The term “computer-readable medium” as used herein refers to any medium that stores data and/or instructions for causing a processor to operate in a specific manner. Such storage medium may comprise non-volatile media and/or volatile media. Non-volatile media may include, for example, optical or magnetic disks. Volatile media may include dynamic memory. Exemplary forms of storage medium include, a floppy disk, a flexible disk, a hard disk, a solid state drive, a magnetic tape, or any other magnetic data storage medium, a CD-ROM, any other optical data storage medium, any physical medium with one or more patterns of holes, a RAM, a PROM, an EPROM, a FLASH-EPROM, NVRAM, and any other memory chip or cartridge.

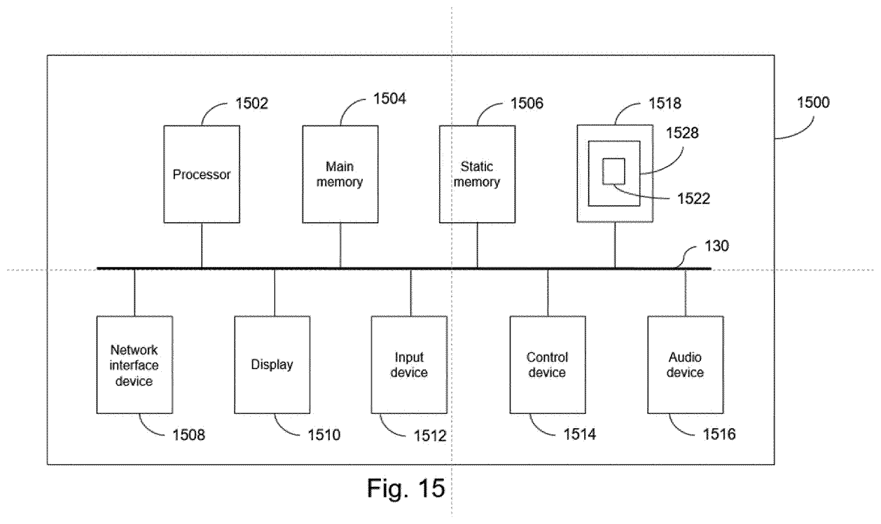

FIG. 15 illustrates a block diagram of one implementation of a computing device 1500 within which a set of instructions, for causing the computing device to perform any one or more of the methodologies discussed herein, may be executed. In alternative implementations, the computing device may be connected (e.g., networked) to other machines in a Local Area Network (LAN), an intranet, an extranet, or the Internet. The computing device may operate in the capacity of a server or a client machine in a client-server network environment, or as a peer machine in a peer-to-peer (or distributed) network environment. The computing device may be a personal computer (PC), an integrated circuit, a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single computing device is illustrated, the term “computing device” shall also be taken to include any collection of machines (e.g., computers) that individually or jointly execute a set (or multiple sets) of instructions to perform any one or more of the methodologies discussed herein.

The example computing device 1500 includes a processing device 1502, a main memory 1504 (e.g., read-only memory (ROM), flash memory, dynamic random access memory (DRAM) such as synchronous DRAM (SDRAM) or Rambus DRAM (RDRAM), etc.), a static memory 1506 (e.g., flash memory, static random access memory (SRAM), etc.), and a secondary memory (e.g., a data storage device 1518), which communicate with each other via a bus 1530.

Processing device 1502 represents one or more general-purpose processors such as a microprocessor, central processing unit, or the like. More particularly, the processing device 1502 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 1502 may also be one or more special-purpose processing devices such as an application specific integrated circuit (ASIC), a field programmable gate array (FPGA), a digital signal processor (DSP), network processor, or the like. Processing device 1502 is configured to execute the processing logic (instructions 1522) for performing the operations and steps discussed herein.

The computing device 1500 may further include a network interface device 1508. The computing device 1500 also may include a video display unit 1510 (e.g., a liquid crystal display (LCD) or a cathode ray tube (CRT)), an alphanumeric input device 1512 (e.g., a keyboard or touchscreen), a cursor control device 1514 (e.g., a mouse or touchscreen), and an audio device 1516 (e.g., a speaker).

The data storage device 1518 may include one or more machine-readable storage media (or more specifically one or more non-transitory computer-readable storage media) 1528 on which is stored one or more sets of instructions 1522 embodying any one or more of the methodologies or functions described herein. The instructions 1522 may also reside, completely or at least partially, within the main memory 1504 and/or within the processing device 1502 during execution thereof by the computer system 1500, the main memory 1504 and the processing device 1502 also constituting computer-readable storage media.

The various methods described above may be implemented by a computer program. The computer program may include computer code arranged to instruct a computer to perform the functions of one or more of the various methods described above. The computer program and/or the code for performing such methods may be provided to an apparatus, such as a computer, on one or more computer readable media or, more generally, a computer program product. The computer readable media may be transitory or non-transitory. The one or more computer readable media could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD.

In an implementation, the modules, components and other features described herein can be implemented as discrete components or integrated in the functionality of hardware components such as ASICS, FPGAs, DSPs or similar devices.

A “hardware component” is a tangible (e.g., non-transitory) physical component (e.g., a set of one or more processors) capable of performing certain operations and may be configured or arranged in a certain physical manner. A hardware component may include dedicated circuitry or logic that is permanently configured to perform certain operations. A hardware component may be or include a special-purpose processor, such as a field programmable gate array (FPGA) or an ASIC. A hardware component may also include programmable logic or circuitry that is temporarily configured by software to perform certain operations.

Accordingly, the phrase “hardware component” should be understood to encompass a tangible entity that may be physically constructed, permanently configured (e.g., hardwired), or temporarily configured (e.g., programmed) to operate in a certain manner or to perform certain operations described herein.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “receiving”, “determining”, “comparing”, “enabling”, “maintaining,” “identifying or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Multiple in-house Python (v3.7) scripts were developed to extract and analyze the data using standard data science packages including: NumPy, Pandas and Scikit-Learn.

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. A computer-implemented method of identifying the presence of any of a plurality of prospective target nucleic acids in a solution containing a biological sample, the method comprising:
receiving amplification curve data indicative of an amplification reaction associated with at least one unknown nucleic acid present in the solution;
processing the received data, wherein the processing comprises inputting input data into a machine learning model trained to identify any of the plurality of prospective target nucleic acids, wherein the input data is based on the amplification curve data and is indicative of the degree of amplification of the at least one unknown nucleic acid over time during the amplification reaction; and
based on the processing, determining that the at least one unknown nucleic acid is one of the plurality of prospective nucleic acids, and thereby identifying the presence of at least one of the plurality of target nucleic acids in the solution.

2. The method of claim 1, wherein the amplification curve data is received from a thermocycler or a device configured to perform an amplification reaction.

3. The method of claim 1, wherein the receiving and the processing occurs in real-time as the amplification reaction is ongoing.

4. The method of claim 1, wherein the amplification curve data and/or the input data comprises a time series depicting the degree of amplification over time throughout a majority of the duration of the amplification reaction.

5. The method of claim 4, wherein the time series depicts the degree of amplification throughout the entirety of the duration of the amplification reaction.

6. The method of claim 4, wherein the amplification curve data and/or the input data comprises a time series depicting the degree of amplification over time from an initial phase in which no amplification is occurring until at least a saturation phase.

7. The method of claim 1, wherein the amplification curve data and/or the input data is representative of an entire amplification curve.

8. The method of claim 1, wherein the amplification curve data is real-time PCR data.

9. (canceled)

10. The method of claim 1, further comprising pre-processing the amplification curve data to generate the input data, wherein pre-processing comprises any of background subtraction, normalization, and artificially increasing the volume of real-time amplification data and/or melting curve data using data augmentation techniques.

11. The method of claim 1, wherein the machine learning model has been trained using labelled amplification curve data, the labelled amplification curve data comprising respective data subsets each associated with a different one of the plurality of prospective target nucleic acids.

12. The method of claim 1, further comprising determining, based on the processing, which of the plurality of prospective target nucleic acids the unknown nucleic acid is most likely to be.

13. The method of claim 1, further comprising receiving melting curve data associated with the at least one unknown nucleic acid, the melting curve data being indicative of a degree of dissociation of the at least one unknown nucleic acid with increasing temperature; and
wherein the input data is further based on the melting curve data.

14. The method of claim 13, wherein the machine learning model has been trained using labelled melting curve data, the labelled melting curve data comprising respective data subsets each associated with a different one of the plurality of prospective target nucleic acids.

15. The method of claim 13, wherein the degree of dissociation of the at least one unknown nucleic acid is determined via monitoring the fluorescence of the solution.

16. The method of claim 14, wherein the solution contains an intercalating dye.

17. The method of claim 13, wherein the input data is combined input data, and wherein the machine learning model is a concluding machine learning model in a system of machine learning models comprising a first, a second, and the concluding machine learning model; wherein processing the received data further comprises:
inputting first input data into the first machine learning model, the first input data being based on the received amplification curve data and the first machine learning model being trained to identify any of the plurality of prospective target nucleic acids based on the first input data;
inputting second input data into the second machine learning model, the second input data being based on the received melting curve data and the second machine learning model being trained to identify any of the plurality of prospective target nucleic acids based on the second input data;
generating the combined input data based on outputs from the first and second machine learning models; and
inputting the combined input data into the concluding machine learning model, the concluding machine learning model being trained to identify any of the plurality of prospective target nucleic acids based on the combined input data.

18. The method of claim 1, wherein the at least one unknown nucleic acid is a plurality of unknown nucleic acids, and the method further comprises determining that each of the plurality of unknown nucleic acids is a member of the plurality of prospective nucleic acids, and thereby identifying the presence of a plurality of different nucleic acids present in the solution.

19. A computer-implemented method of training a machine learning model to identify any of a plurality of prospective target nucleic acids in a solution comprising a biological sample, the method comprising:
receiving amplification curve data indicative of an amplification reaction associated with at least one known nucleic acid, the known nucleic acid being one of the plurality of prospective target nucleic acids;
processing the received data, wherein the processing comprises inputting input data into a machine learning model to generate a prediction as to whether the known nucleic acid is one of the plurality of prospective target nucleic acids, wherein the input data is based on the amplification curve data, is indicative of the degree of amplification of the at least one known nucleic acid over time, and is labelled according to the known nucleic acid; and
based on the generated prediction, training the machine learning model to identify any of the plurality of prospective target nucleic acids.

20. The method of claim 19, further comprising receiving melting curve data associated with the at least one known nucleic acid, the melting curve data being indicative of a degree of dissociation of the at least one known nucleic acid with increasing temperature; and
wherein the input data is further based on the melting curve data.

21. A computer readable medium comprising computer executable instructions which, when performed by a processor, cause the processor to perform the a method of identifying the presence of any of a plurality of prospective target nucleic acids in a solution containing a biological sample, the method comprising:
receiving amplification curve data indicative of an amplification reaction associated with at least one unknown nucleic acid present in the solution;
processing the received data, wherein the processing comprises inputting input data into a machine learning model trained to identify any of the plurality of prospective target nucleic acids, wherein the input data is based on the amplification curve data and is indicative of the degree of amplification of the at least one unknown nucleic acid over time during the amplification reaction; and
based on the processing, determining that the at least one unknown nucleic acid is one of the plurality of prospective nucleic acids, and thereby identifying the presence of at least one of the plurality of target nucleic acids in the solution.
